(19) United States
(12) Patent Application Publication
(10) Pub. No.: US 2016/0121096 A1
(43) Pub. Date: May 5, 2016

(54) APPARATUS AND SYSTEMS FOR MEDICAL DEVICES AND CORRESPONDING INTERFACE UNITS AND METHODS FOR PRODUCING AND OPERATING THE SAME

(71) Applicant: Q-CORE MEDICAL LTD., Netanya (IL)

(72) Inventors: Shachar Rotem, Kibbutz Metzer (IL); Michael Nikolaevsky, Rishon Lezion (IL); Amir Rasowsky, Yakir (IL)

(21) Appl. No.: 14/527,791

(22) Filed: Oct. 30, 2014

(51) Int. Cl. A61M 39/10; A61M 5/50; A61M 5/142; A61M 5/172; A61M 39/12

(52) U.S. Cl. CPC A61M 39/1011; A61M 5/172; A61M 39/12; A61M 5/14228; A61M 5/5086; A61M 2039/1094; A61M 2205/123; A61M 2039/1072; A61M 2205/0272; A61M 2205/33

(57) ABSTRACT

Disclosed is a medical device including a housing which may receive an interface unit body which may include at least one flexible segment, a therapeutic component which may apply a force to cause fluid to flow through the flexible segment and a sensor to (1) detect if the interface unit body corresponds to said housing and (2) disable said therapeutic component if the interface unit body is non-corresponding.

### FIELD OF THE INVENTION

The present invention relates generally to the field of medical devices. More specifically, the present invention relates to medical devices and corresponding interface units and methods for producing and/or operating the same.

### BACKGROUND

Various types of medical devices are known and used in hospitals, home settings and the like in the treatment of patients. Some medical devices cause fluid to flow through the medical device or associated sets and/or interface units. An example of this type of medical device may be a medical infusion pump, dialysis machine and more.

Various types of medical infusion pumps are known in the art. One common type of infusion pump is a peristaltic pump, in which fluid is made to flow through an elastic tube or a conduit by external compression of the tube or a flexible segment of a conduit. A peristaltic mechanism, such as a set of cams or fingers, may compress the tube in a cyclic pattern at a sequence of locations along the length of the tube or the flexible segment of the conduit, so as to cause the fluid to flow through the tube/conduit at a desired volumetric rate.

When incorporated into medical systems, peristaltic pumps are often used to pump liquids within an infusion system. The relative accuracy and controllability of such pumps makes them desirable for such implementations, as the administration of pharmaceutical liquids to patients requires accuracy and strict regulation of flow. In such implementations, the pumping mechanism may be reusable, whereas the infusion tubes/sets/cassettes and/or conduits may be disposable. Accordingly, it is necessary to interface between the infusion tubes and/or conduits and the pump. It is therefore desirable to provide pump to infusion tube interfaces which promote accuracy and reliability of the system which are yet easy to use. In some embodiments the interface and the tubing are separate while in some cassettes and sets the conduit is integrated within the interface.

### SUMMARY OF THE INVENTION

The present invention includes apparatus and systems for medical devices and corresponding interface units and methods for producing and operating the same. According to some embodiments, an apparatus may be engaged with a medical device, the medical device may include a therapeutic component and at least one sensor, the apparatus may include: an interface unit body including at least one flexible segment to receive a force from the therapeutic component to cause fluid to flow through the flexible segment, and a sensor-interfacing structure configured to: (a) enable engagement with a corresponding medical device and/or (b) automatically disable the therapeutic component of the medical device when the interface unit body is connected to a non-corresponding medical device.

According to some embodiments, a medical device may include a housing to receive an interface unit body which may include at least one flexible segment, a therapeutic component which may apply a force to cause fluid to flow through the flexible segment, and a sensor which may (1) detect if the interface unit body corresponds to the housing and/or (2) disable the therapeutic component if the interface unit body is non-corresponding.

According to some embodiments, the sensor-interfacing structure may be clasped onto the apparatus. The flexible segment may be a silicon tube and/or a reservoir.

According to some embodiments, the interface unit body may include at least one of the elements selected from the list consisting of: a gasket, a piston, a hinge, a cam, a wheel and sealing membranes.

According to some embodiments, the therapeutic component may include substantially non-flexible fluid conduits.

According to some embodiments, the sensor-interfacing structure may include a rigid element which may be configured to obstruct a door of a non-corresponding medical device from closing. The rigid element may be configured to enable a door of a corresponding medical device to close.

According to some embodiments, the medical device may include a keying mechanism to cause the sensor to detect a non-corresponding interface unit body as non-corresponding.

According to some embodiments the medical device may include a rim surrounding the housing and a door to close over the rim and to prevent unintentional detachment of the interface unit body from the housing. The medical device may also include a door and the sensor may be configured to detect that the interface unit is non-corresponding when the door is unlatched/at least partially open. The sensor may be a magnetic sensor. The therapeutic component may be a peristaltic pumping mechanism including one or more fingers to apply the force.

According to some embodiments, a method of operating a peristaltic pump may include: installing an interface unit in conjunction with the peristaltic pump, detecting if the interface unit is corresponding or non-corresponding to the peristaltic pump and disabling a therapeutic component of the peristaltic pump if the interface is non-corresponding.

According to some embodiments, the interface unit may be detected as non-corresponding if a door associated with the peristaltic pump is sensed as being open. According to some embodiments, the interface unit may be detected as non-corresponding if an opening in the peristaltic pump is sensed as being non-obstructed/non-full. According to some embodiments, a warning/alarm may be emitted if the interface unit is detected as non-corresponding.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A & 1B are block level diagrams of example medical devices each in conjunction with an example interface unit body, all in accordance with some embodiments of the present invention;

FIG. 2 is a block level diagram of an example interface unit body in conjunction with an example medical device, all in accordance with some embodiments of the present invention;

FIG. 3 is a block level diagram of an example medical system including an example interface unit body and an example medical device, all in accordance with some embodiments of the present invention;

FIG. 4 is a block level schematic of a plurality of different example medical devices and different example interface units in accordance with some embodiments of the present invention;

FIG. 5 is a flow chart of an example method in accordance with some embodiments of the present invention; and

FIGS. 6A and 6C are top views of example interface units in accordance with some embodiments of the present invention;

FIGS. 6B and 6D are side views of the example interface units of FIGS. 6A and 6C (respectively) in accordance with some embodiments of the present invention;

FIG. 7 is an example medical device including a door shown in an open position in accordance with some embodiments of the present invention; and

FIG. 8 is an example top view of example doors in accordance with some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, electronic circuit(s), or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The present invention includes methods, circuits, apparatuses and systems for selectively enabling operation of an interface unit with a medical device. According to some embodiments, a first type of medical device may correspond to one or more types of interface units. The medical device and/or each type of corresponding/matching/correlating interface unit may be configured so that a corresponding interface unit may be placed in conjunction with the medical device. According to some embodiments, if a non-corresponding/mismatched/non-correlating interface unit is placed at least partially in conjunction with a medical device then the medical device may be disabled.

According to some embodiments a medical device may include a therapeutic component to carry out a therapeutic functionality. According to some embodiments, the medical device may include a sensor configured to detect when a non-corresponding interface unit is placed at least partially in conjunction with the medical device and/or confirm/detect that a corresponding interface unit is connected to the medical device thus enabling the therapeutic component of the medical device to be enabled. According to some embodiments, depending on the mechanical configuration of a specific embodiment, the non-corresponding interface unit may be placed in conjunction with the medical device or only partially in conjunction since in some embodiments the mechanical configuration may not enable placing a non-corresponding interface unit completely in conjunction with a medical device. According to some embodiments, the medical device may include a keying mechanism which may at least partially cause a non-corresponding interface unit not to be able to be placed completely in conjunction with the medical device.

According to some embodiments, an interface unit may include at least a flexible segment. The flexible segment may be configured to receive a force from a corresponding medical device to cause fluid to flow through said flexible segment. The interface unit may further include a sensor interfacing structure/element to (a) enable engagement with a corresponding medical device and (b) automatically disable the therapeutic component of the medical device when said interface unit body is connected to a non-corresponding medical device.

According to some embodiments, a medical device may include a keying segment/mechanism. One or more types of medical devices may be differentiated by the keying segment. The keying segment may be part of a door, a latch, an interface housing or otherwise. According to some embodiments, the keying segment may be selected so that production of the different medical devices is such that determining if the medical device with be of a first, second, third type or otherwise is determinable at a substantially final stage of the production and/or changeable by a technician. Furthermore, the keying segment may be independent of the therapeutic component so that switching between a first or second type of medical device does not require safety or regulatory testing or authorization.

According to some embodiments, an interface unit may include a sensor-interface segment so that a first type of interface unit may be connectable/placed in conjunction with a first type of corresponding medical device the medical device may operate with the first type of interface unit. A first type of interface unit may not be able to be placed in conjunction with a non-corresponding medical device and/or if placed at least partially in conjunction with a non-corresponding medical device then the medical device may be disabled. A first type of interface unit may be differentiated with a second type by either having different types of sensor-interface segments and/or lacking a sensor-interface segment. A sensor-interface segment may be mechanically latched to an interface unit or they may be produced together, for example using a plastic mold. Examples of sensor-interface segments may include: a mechanical segment optionally made of a plastic material, an optical segment (such as a QRcode or otherwise), a magnetic segment (such as a magnetic key) and more.

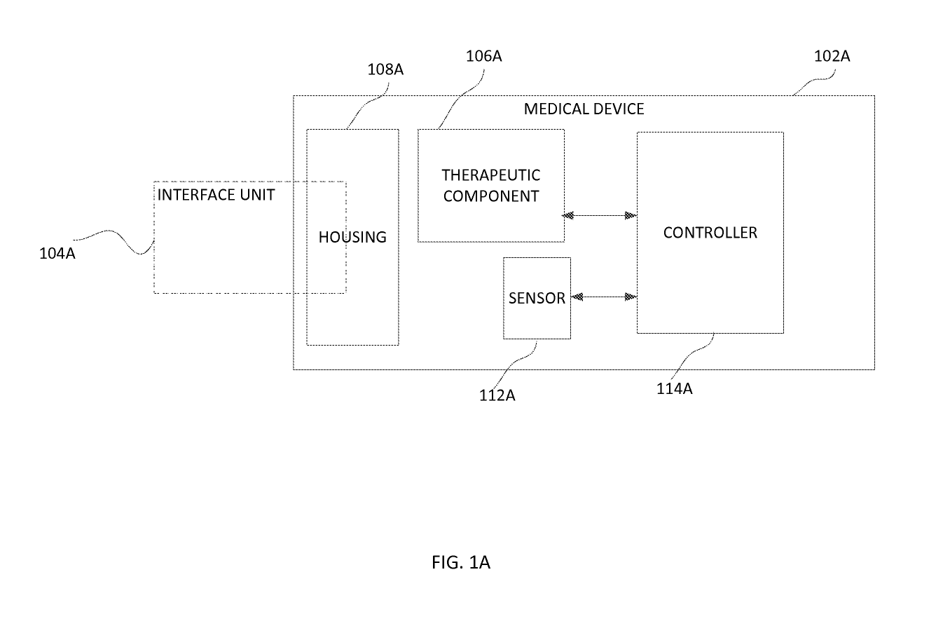

Turning to FIG. 1A, depicted is a block level diagram of an example medical device (102A) which may operate in conjunction with an example interface unit body (104A). Medical device 102A may be a medical pump such as a peristaltic pump, syringe pump and more and/or additional types of medical devices such as a dialysis machine, heart and lung machine and additional types of medical devices which may include a connectable and detachable segment. The connectable and detachable segment may be referred to as a set. In some examples the set associated with a medical device may be at least partially disposable while the medical device itself may be used a plurality of times. In yet further embodiments, the medical device itself may also be disposable and/or the set may be reusable. Optionally, the interface unit may be utilized to cause fluids to flow through them while medical device 102A does not come in direct contact with medication and/or human fluids so that medical device 102A can be re-used many times and does not require substantial sterilization between treatments or the like.

According to some embodiments, medical device 102A may include a therapeutic component (106A) to carry out a therapeutic functionality. For example, if medical device 102A is a medical pump, therapeutic component 106A may include a peristaltic pumping mechanism such as actuators and/or fingers and if medical device 102A is a dialysis machine, therapeutic component 106A may include a filtering mechanism and/or fluid pumping mechanism. Additional therapeutic components for medical device 102A are understood.

According to some embodiments, medical device 102A may include a housing 108A to receive an interface unit. Housing 108A may include a door which may be configured to lock/secure an interface unit in the medical device. Housing 108A may include at least a part of anti-free flow mechanism to ensure that fluid (such as medication or patient associated fluids) cannot flow in an uncontrolled manner which may be important for therapeutic safety reasons. Housing 108A may include latches and additional elements to secure/lock an interface unit. Housing 108A may include openings and/or access points/sections so that therapeutic component 106A may operate on interface unit body 104A when it is placed in conjunction with housing 108A.

According to some embodiments, medical device 102A may include a sensor (112A). Sensor 112A may be a magnetic sensor, mechanical sensor, electronic sensor, infra-red sensor, optical sensor or otherwise. Sensor 112A may be configured to detect/sense that a non-corresponding interface unit is at least partially placed in conjunction with medical device 102A. Sensor 112A may be integral with housing 108A or in any section of medical device 102A.

According to some embodiments, medical device 102A may include a controller (114A). Controller 114A may be configured to activate and/or receive information, feedback and the like from therapeutic component 106A. Controller 114A may receive information/a signal from sensor 112A to indicate if a corresponding and/or non-corresponding interface unit is placed in conjunction with medical device 102A. Controller 114A may disable or enable therapeutic component 106A at least partially dependent on a signal/indication received from sensor 112A. If a non-corresponding interface unit is placed in conjunction with medical device 102A, sensor 112A may disable the therapeutic component. Sensor 112A may signal a controller (114A) which may then disable therapeutic component 106A.

Turning to FIG. 1B, it is understood that elements 102B-114B are substantially similar to elements 102A-114A of FIG. 1A. According to some embodiments, medical device 102B may further include a keying mechanism (150B) which may be integral to or associated with housing 108B. Keying mechanism 150B may be configured to enable selective connection of appropriate and/or corresponding interface units. According to some embodiments, keying mechanism 150B may interfere with a non-corresponding interface unit from being placed in conjunction with medical device 102B. Optionally, there may be one or a plurality of corresponding interface unit types and/or one or a plurality of non-corresponding interface unit types.

According to some embodiments, keying mechanism 150B may be connected mechanically (for example: clasped on, screwed on and the like), may be connected chemically for example with an adhesive and/or may be integral to the medical device (for example part of a plastic mold or welded on and more) or otherwise.

According to some embodiments, keying mechanism 150B may be any part of medical device 102B such as a door, AFFV (anti free flow) mechanism, latch and more. Keying mechanism 150B may be of a male/female configuration having a specific shape/opening/protrusion so that an interface unit with a complementing shape may be placed in conjunction with medical device 102B.

According to some embodiments, keying mechanism 150B may be non-dependent or not affect the therapeutic functionality of medical device 102B. Keying mechanism 150B may not be in-line to the therapeutic functionality and accordingly existence or non-existence of keying mechanism 150B may be designed/selected to not affect the therapeutic functionality and/or parameters, such as: flow rate, accuracy, bubble detection, error detection, safety features and/or more.

According to some embodiments, keying mechanism 150B may be configured to effect sensor 112B resultant signals. Keying mechanism 150B may cause sensor 112B to detect interface unit 104B as being non-corresponding to medical device 102B if interface unit body 104B is placed at least partially in conjunction with a non-corresponding medical device.

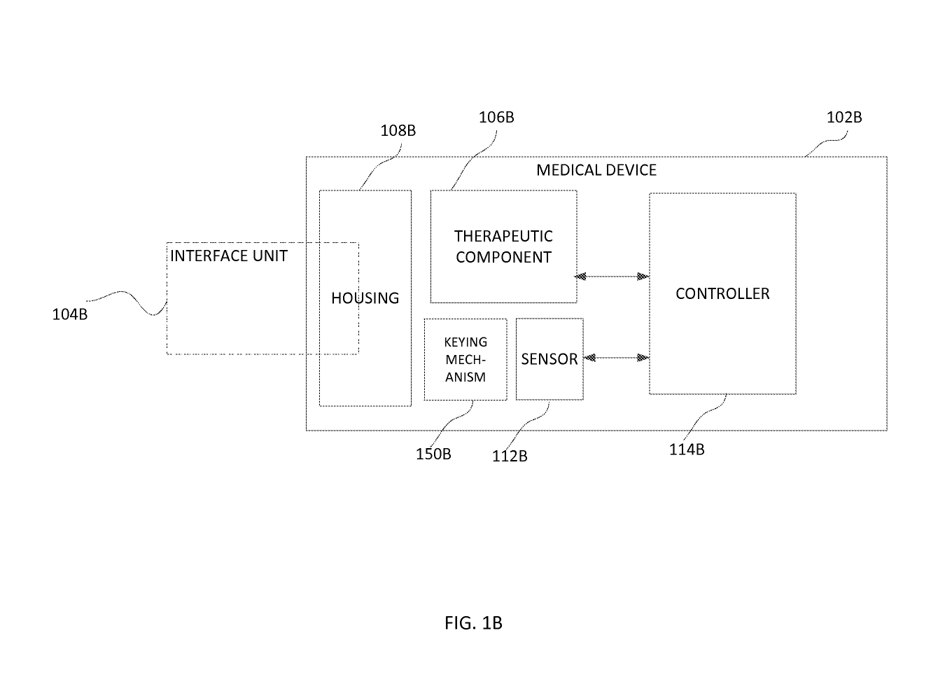

Turning to FIG. 2, depicted is a block level diagram of an example interface unit (204) which may be placed at least partially in conjunction with an example medical device (202). Elements 206-214 of medical device 202 are substantially similar to elements 106A-114A of medical device 102A. According to some embodiments, interface unit 204 and interface unit 104A are substantially similar.

Interface unit 204 may be a cassette, a set, a plastic housing, an interface to connect a tube to a medical device and more. Interface unit 204 may include an embedded tube (silicon, plastic or otherwise) or may have a section to receive at least a flexible segment or may include: an at least partially rigid tubing, gaskets, reservoirs and more. Interface unit 204 may include a flexible segment (216) to receive a force from therapeutic component 206 to cause fluid to flow through flexible segment 216.

According to some embodiments, the force from therapeutic component 206 may be received from one or more peristaltic fingers, magnetic actuators and more.

According to some embodiments, interface unit (204) may include a sensor interfacing structure (218). Sensor-interfacing structure 218 may enable engagement of interface unit 204 with housing 208. Sensor-interfacing structure 218 may automatically cause sensor 212 to disable therapeutic component 206 of medical device 202 if medical device 202 is non-corresponding to interface unit 204. Sensor-interfacing structure may include a keying segment to interact/respond/relate to keying mechanism 250 so that correlating interface units and medical devices may operate in conjunction, while non-correlating medical device and interface units are disabled.

According to some embodiments, sensor-interface structure 218 may be an integral or added-on element to interface unit 204. Sensor interface structure 218 may be adhered, clasped and/or mechanically or chemically connected to sensor interface structure 218 and more or otherwise. Accordingly, sensor-interface structure 218 may be added as a final stage in production or may be an internal step in production or otherwise. Sensor interface 218 may be a mechanical segment, a magnetic element (such as a magnetic key), an optical element (such as a QRcode) and more. Sensor interface 218 may be matched to sensor 212 so that sensor 212 may detect if interface unit 216 is corresponding based on sensor-interface 218.

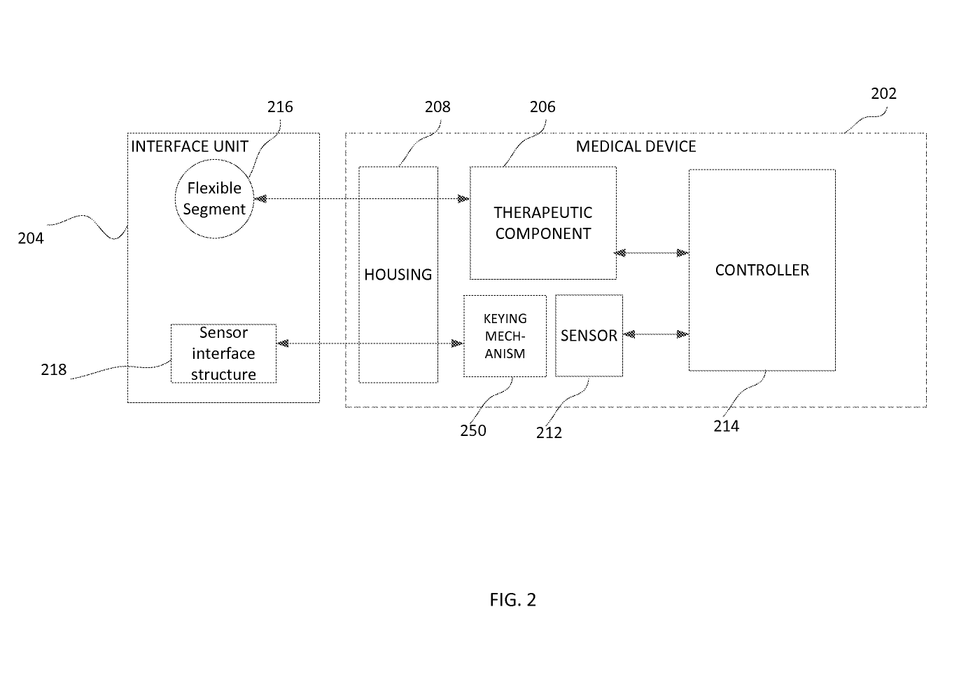

Turning to FIG. 3, depicted is a block level diagram of an example medical system including an example interface unit body and an example medical device. It is understood that medical device 302 is substantially similar to medical devices 102A and 202 and that interface unit 304 is substantially similar to interface units 104A and 204. Accordingly, elements 306-318 are substantially similar to elements 206-218 of FIG. 2.

According to some embodiments, medical device 302 may operate on interface unit 304 if interface unit is placed in conjunction with medical device 302 and corresponds to medical device 302. If interface unit 304 is non-corresponding, sensor 312 may detect interface unit 304 as such and cause therapeutic component 306 to be disabled.

According to some embodiments, if interface unit 304 is non-corresponding it may be placed partially in conjunction with medical device 302, thus causing sensor 312 to detect interface unit 304 as non-corresponding.

According to some embodiments, medical device 302 may optionally include keying mechanism 350. According to some embodiments, sensor-interface structure 318 may be of a male/female configuration having a specific shape/opening/protrusion so that if interface unit 304 has a complementing shape to keying mechanism 350 it may be placed in conjunction with medical device 302.

According to some embodiments, keying mechanism 350 may be configured so that a non-corresponding interface unit may not be placed in conjunction and/or may not be operated upon by medical device 302. This feature may enhance medical safety of medical device 302 to limit/decrease connection of improper interface units being engaged with medical device 302 by a user.

According to some embodiments, medical device 302 may include a plurality of sensors 312 and may be configured to detect a plurality of interface units 304 as corresponding and/or non-corresponding. Accordingly each interface unit 304 may have one or more sensor-interface structures (318) so that a plurality of corresponding and differentiating medical devices and corresponding interface units may be achieved.

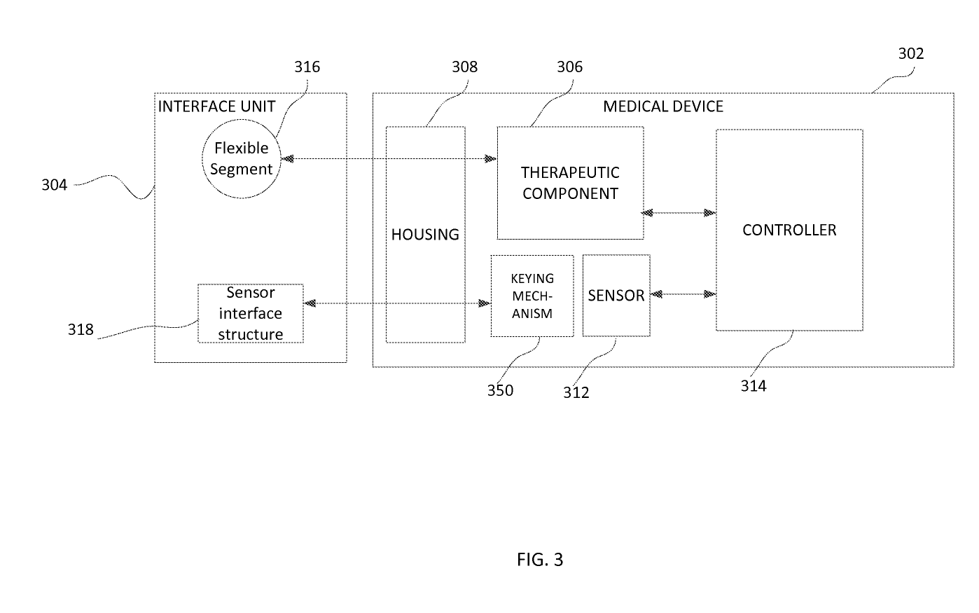

Turning to FIG. 4, depicted is a block level schematic (400) of a plurality of different example medical devices and different example interface units in accordance with some embodiments of the present invention. According to some embodiments, medical device 402A and 402B may each be similar to medical device 302 or only one of medical devices 402A and 402B may be similar to medical device 302. Optionally, medical device 402A and 402B may be differentiated in that each is of a different type, each has a different keying mechanism and/or one has a keying mechanism while the other lacks a keying mechanism and more. It is understood that medical device 402A and/or 402B may both lack a keying mechanism, as a keying mechanism is an optional element according to some embodiments.

According to some embodiments, one or more of interface units 404A-404C may be similar to interface unit 304. Optionally, interface units 404A-404C may be differentiated in that each is of a different type, each has a different sensor interfacing structure and/or one or more has a sensor interfacing structure while one or more lack a sensor interfacing structure or otherwise.

According to some embodiments, as depicted, medical device 402A may be operable with interface units 404A and/or 404C. Accordingly, medical device 402A may be determined to correspond to either interface unit 404A and/or 404C if either is placed in conjunction with medical device 402A. Medical device 402A may be determined not to correspond (or to be non-corresponding) to interface unit 404B, accordingly, if interface unit 404B is placed at least partially in conjunction with medical device 402A, a therapeutic component of medical device 402A may be disabled.

According to some embodiments, interface unit 404B may be determined to correspond to medical device 402B and accordingly, and may be operable in conjunction to/with medical device 402B. Similarly, interface unit 404A may also correspond to medical device 402B while interface unit 404C is non-corresponding to medical device 402B.

While two example medical devices (402A and 402B) and three example interface units (404A-404C) are shown in this example, it is understood that any combination of different types of medical devices and/or interface units is applicable.

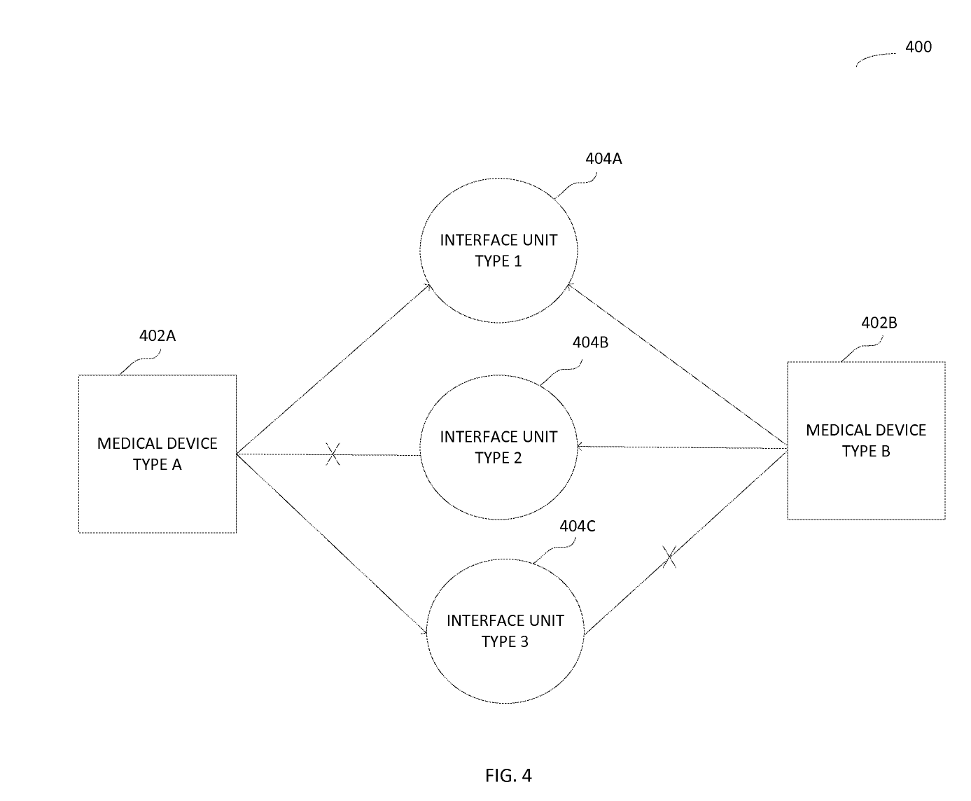

Turning to FIG. 5, depicted is a flow chart of an example method (500). According to some embodiments, an interface unit may be installed at least partially in conjunction with a medical device (step 502); it is then analyzed/determined if the interface unit is non-corresponding (step 504). If the interface unit and the medical device are non-corresponding to each other the medical device (or a therapeutic component of the medical device) may be disabled (step 506). If the interface unit and medical device are corresponding the medical device (or a therapeutic component of the medical device) may be enabled (step 508).

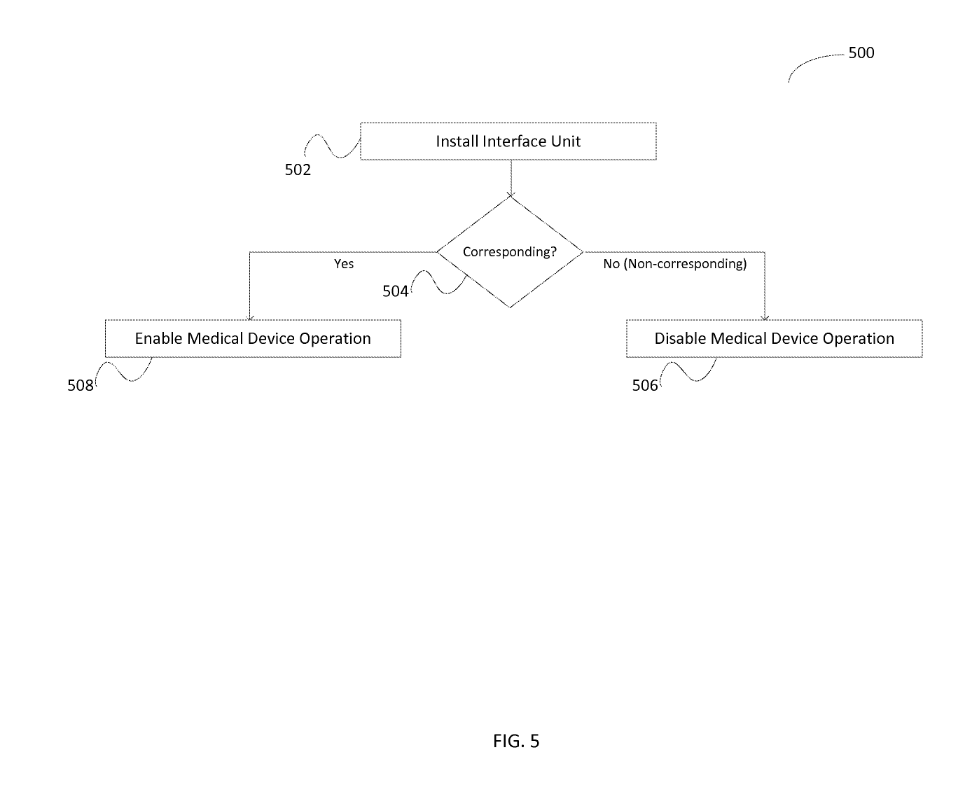

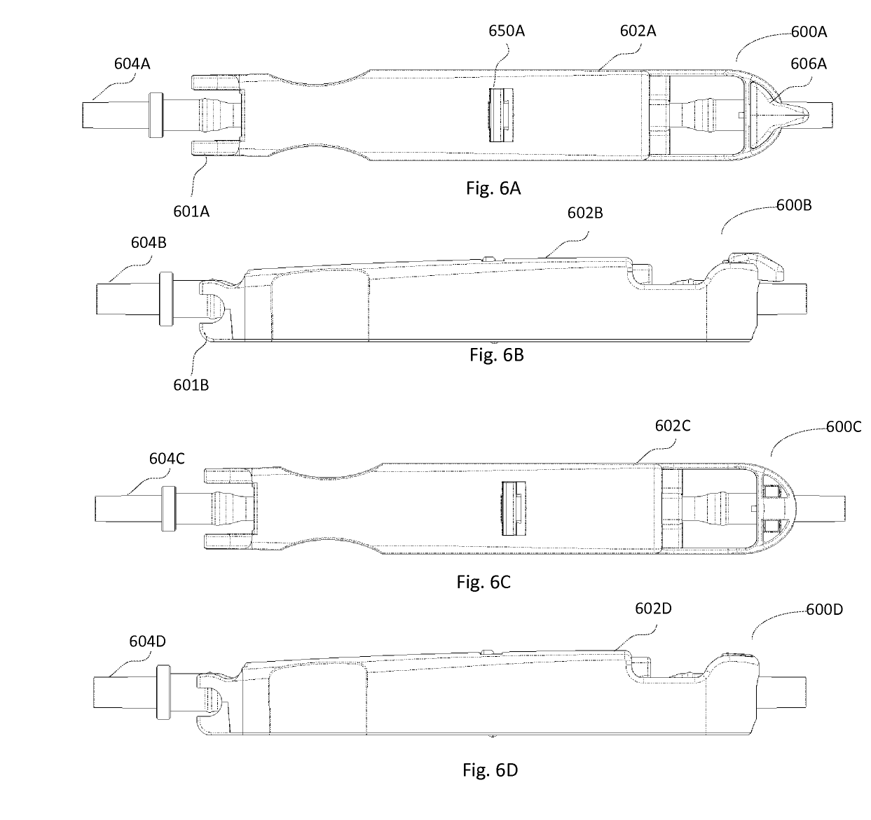

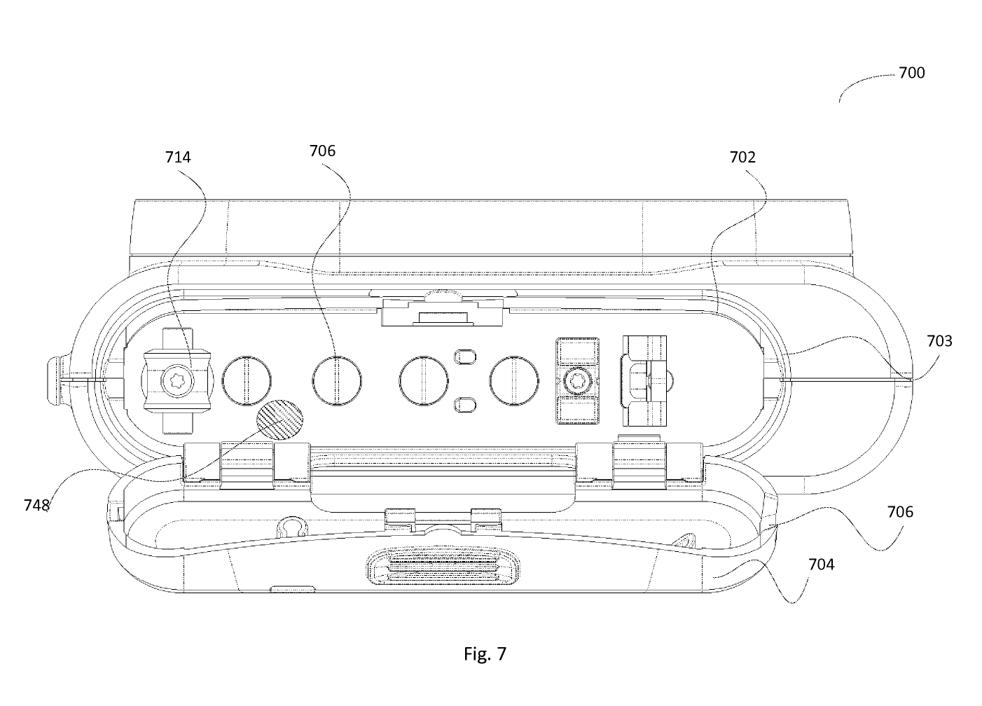

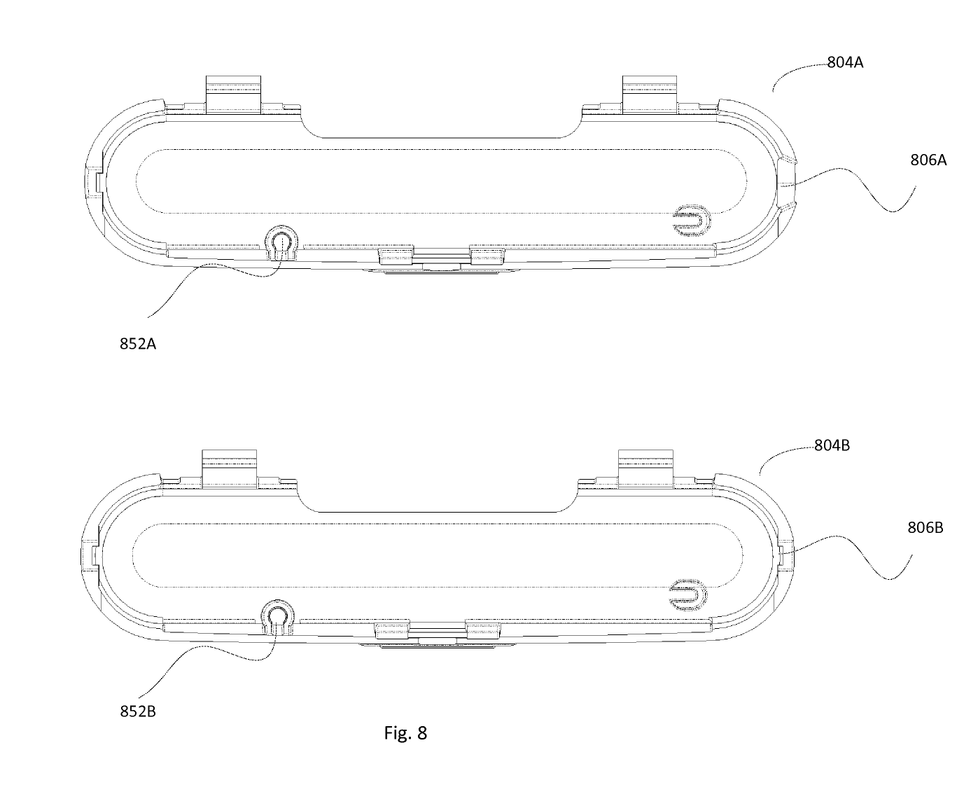

Turning to FIG. 6A-6D, FIG. 7 and FIG. 8, interface unit 600A may include a saddle-like catch (saddle 601A), located e.g., at the downstream end of the interface unit 602A; medical device 700 may include a barrel-like axle (barrel 714) which may be configured to fit saddle 601A by means of shape and size such that a two-parts hinge is provided at time saddle 601A and barrel 800 are integrated.

According to some embodiments, interface unit 602A may include pistons, hinges, cams, wheels, sealing membranes, gaskets and more. Or may be a passive interface (as depicted in FIG. 6A-6D) and include only static elements such as tubing 604A in a static configuration.

According to some embodiments, the interface unit 602A may include one or more replaceable mechanical subunits so that by replacing/adding one or more subunits, correlation to medical device 700 is determined. Accordingly, protrusion 606A may be a sensor interface structure.

FIG. 8 depicts 2 example doors with different openings 806A and 806B which may replace door 704, it is understood that protrusion 606A may fit into opening 806A and may not fit into opening 806B. It is understood that either door or additional alternative door configurations may be assembled onto medical device 700.

According to some embodiments, medical device 700 may include a door (704) including opening 706. Both interface units 602A and 602C may fit into housing 702. Door 704 may include an opening 706 so that when a corresponding interface unit is placed in conjunction with housing 702 door 704 can completely shut. A sensor associated with medical device 700 may detect that door 704 is completely shut and enable a therapeutic component of medical device 700 to operate. The sensor may be located below housing 702 as indicated by sensor location 748 in the figure. If a non-corresponding interface unit is placed in conjunction with housing 702, opening 706 may be too small for protrusion 606A to be placed through; accordingly door 704 may not completely shut. The associated sensor may detect that door 704 is not completely shut, and cause a therapeutic component of medical device 700 to be disabled.

According to some examples, door 806A or 806B may replace door 704. A sensor included in medical device 700 may be a magnetic sensor located under 748 and may sense when a magnet (852A or 852B, appropriately) is near the sensor and accordingly detect that the associated door (806A or 806B appropriately) is closed or open.

According to some embodiments, depending on the geometrics of protrusion 606A and opening 706; both interface unit 602A and 602C may be placed in conjunction with the same medical device. According to other embodiments with different geometrics only interface 602C may be mechanically able to be placed in conjunction with medical device 700.

According to another embodiment of the present invention wherein the two or more replaceable mechanical subunits (protrusion 606A and door 804) may be interconnected in a male-female fitting mechanism. It is according to another embodiment of the present invention wherein the system as defined in any of the above comprises a master keying mechanism, wherein at least one interface unit type is adapted to be utilized in a plurality of all medical devices.

It is according to another embodiment of the present invention the master keying mechanism (i.e. door 704) is adapted to match at least one interface unit (600A and/or 600C) of a given characterization with a plurality of N predetermined medical devices; N is any integer number, selected from either defined or not defined number of medical device possible combinations, in this example the medical devices vary depending on door configuration (such as door 804A and 804B).

According to some embodiments, magnets 852A and 852B may vary in size, type, location, strength or otherwise or may be identical. In an alternative embodiment, magnet 852A may be located on interface unit 600A so that sensor and may be included in sensor interface structure. A magnet 852B may be located on interface unit 600C so that a sensor associate with the medical device may differentiate between a corresponding interface structure based on strength, location, polarity or otherwise of magnet 852A and/or 852B.

According to some embodiments, medical device 700 may include additional elements such as: rim 703 to surround housing 702 and to operate in conjunction with door 704 to close over rim 703 and to prevent unintentional detachment of the interface unit from housing 702, AFFV mechanism to reduce risk of unintentional flow through interface unit while detached from the housing (some elements of the AFFV may include AFFV segment 650A), finger/actuator openings (706) to cause fluid to flow at least through a segment of an interface unit and more.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

What is claimed:

1. An apparatus to be engaged with a medical device, the medical device including a therapeutic component and at least one sensor, said apparatus comprising:
an interface unit body including at least one flexible segment to receive a force from the therapeutic component to cause fluid to flow through said flexible segment; and
a sensor-interfacing structure configured to: (a) enable engagement with a corresponding medical device and (b) automatically disable the therapeutic component of the medical device when said interface unit body is connected to a non-corresponding medical device.

2. The apparatus according to claim 1, wherein said sensor-interfacing structure is clasped onto said apparatus.

3. The apparatus according to claim 1, wherein said flexible segment is a silicon tube.

4. The apparatus according to claim 1, wherein said flexible segment is a reservoir.

5. The apparatus of claim 4, wherein said interface unit body includes at least one of the elements selected from the list consisting of: a gasket, a piston, a hinge, a cam, a wheel and sealing membranes.

6. The apparatus of claim 4, wherein said therapeutic component includes substantially non-flexible fluid conduits.

7. The apparatus of claim 3, wherein said sensor-interfacing structure includes a rigid element configured to obstruct a door of a non-corresponding medical device from closing.

8. The apparatus of claim 7, wherein said rigid element is configured to enable a door of a corresponding medical device to close.

9. A medical device comprising:
a housing to receive an interface unit body including at least one flexible segment;
a therapeutic component to apply a force to cause fluid to flow through said flexible segment;
a sensor to (1) detect if the interface unit body corresponds to said housing and (2) disable said therapeutic component if the interface unit body is non-corresponding.

10. The medical device of claim 9, further comprising a keying mechanism to cause said sensor to detect a non-corresponding interface unit body as non-corresponding.

11. The medical device of claim 9, further comprising a rim surrounding the housing and a door to close over the rim to prevent unintentional detachment of the interface unit body from said housing.

12. The medical device of claim 9, further comprising a door and wherein said sensor is configured to detect that said interface unit is non-corresponding when said door is unlatched/at least partially open.

13. The medical device of claim 12, wherein said sensor is a magnetic sensor.

14. The medical device of claim 9, wherein said therapeutic component is a peristaltic pumping mechanism including one or more fingers to apply said force.

15. A method of operating a peristaltic pump comprising:
installing an interface unit in conjunction with the peristaltic pump;
detecting if said interface unit is corresponding or non-corresponding to said peristaltic pump;
disabling a therapeutic component of said peristaltic pump if said interface is non-corresponding.

16. The method according to claim 15, wherein said interface unit is detected as non-corresponding if a door associated with said peristaltic pump is sensed as being open.

17. The method according to claim 15, wherein said interface unit is detected as non-corresponding if an opening in said peristaltic pump is sensed as being non-obstructed.

18. The method according to claim 15, further comprising emitting a warning if said interface unit is detected as non-corresponding.
